(19) United States
(12) Patent Application Publication
(10) Pub. No.: US 2017/0055915 A1
(43) Pub. Date: Mar. 2, 2017

(54) METHOD AND SYSTEM FOR DETECTING PAIN OF USERS

(71) Applicants: Lakshya JAIN, Los Altos, CA (US); Priya BISARYA, San Diego, CA (US)

(72) Inventors: Lakshya JAIN, Los Altos, CA (US); Priya BISARYA, San Diego, CA (US)

(21) Appl. No.: 14/835,723

(22) Filed: Aug. 26, 2015

(51) Int. Cl. A61B 5/00; A61B 5/16; A61B 90/00; A61B 5/11; A61B 5/08; A61B 5/0402

(52) U.S. Cl. CPC A61B 5/7275; A61B 5/08; A61B 5/0402; A61B 90/39; A61B 5/1102; A61B 5/165

(57) ABSTRACT

The present disclosure provides a method and system for detecting intensity of pain experienced by one or more users. The computer implemented method includes determining, with a processor, the intensity of pain experienced by each of the one or more users by placing each of one or more bio-sensors at one or more locations on body of each of the one or more users; analyzing, with the processor, the determined intensity of pain; recognizing, with the processor, a correlation between the intensity of pain determined from each of one or more bio-markers associated with each of the one or more users and the one or more locations of each of the one or more bio-sensors with respect to locus of the pain of each of the one or more users; and generating, with the processor, a pain scale for each of the one or more users.

### TECHNICAL FIELD

The present invention relates to the field of pain monitoring and, in particular, relates to measuring intensity of pain of users by placing sensors at different locations on their body.

### BACKGROUND

Pain is an unpleasant sensory and emotional experience associated with actual or potential tissue damage. In fact, the pain is a stressor and environment challenge that requires an organism to respond. It is a specific emotion, caused by a stimulus that reflects homeostatic behavioral drive, similar to temperature, itching, hunger, thirst and the like. It may be categorized according to various factors, including type of damage, time for healing and the like. On the basis of healing, the pain can be categorized as a chronic pain and an acute pain. The chronic pain lasts for a longer time as compared to the acute pain. However, both the chronic pain and the acute pain are extremely important problems leading to loss of working capabilities, financial resources and the like.

Experience of the pain varies from person to person due to inter-individual variability. Moreover, intensity of the pain varies from cause to cause in the individual. Thus, pain management is an extremely important issue. Sensors measure the intensity of pain experienced by the organisms. However, it is seen that the intensity of the pain measured by the sensors may vary due to various reasons. One reason isdistance of the pain area from location of the sensors. Thus, it becomes difficult to treat the pain of the organisms effectively.

Presently, there is no adequate method and system that studies relationships between the pain experienced by the organisms and the sensors to guide appropriate treatment, care, lifestyle and the like for healthy response in individuals with respect to the pain. Moreover, the present methods and systems do not reduce/treat the pain effectively in the individuals.

In light of the above stated discussion, there is a need for a method and system that overcomes the above stated disadvantages.

### SUMMARY

In an aspect of the present disclosure, a computer-implemented method for detecting intensity of pain experienced by one or more users is provided. The computer implemented method includes determining, with a processor, the intensity of pain experienced by each of the one or more users by placing each of one or more bio-sensors at one or more locations on body of each of the one or more users; analyzing, with the processor, the determined intensity of pain from readings of one or more bio-markers obtained by placing each of the one or more bio-sensors at the one or more locations on the body of each of the one or more users; recognizing, with the processor, a correlation between the intensity of pain determined from each of the one or more bio-markers associated with each of the one or more users and the one or more locations of each of the one or more bio-sensors with respect to locus of the pain of each of the one or more users; and generating, with the processor, a pain scale for each of the one or more users. The intensity of pain is determined from the one or more bio-markers associated with each of the one or more users. The analyzing is done for determining sensitivity to pain at the one or more locations on the body of each of the one or more users. The correlation reveals a change in the readings of each of the one or more bio-markers for each of the one or more locations of each of the one or more bio-sensors. The recognizing is done for determining an epicenter of pain for each of the one or more users. The pain scale is generated based on the recognizing of the correlation. The pain scale relates distance between the one or more bio-sensors and the locus of the pain with the change in the readings of each of the one or more bio-markers for each of the one or more locations of each of the one or more bio-sensors.

In an embodiment of the present disclosure, the computer-implemented method further includes storing the readings of each of the one or more bio-markers and the generated pain scale for each of the one or more users.

In an embodiment of the present disclosure, the one or more bio-sensors include a ballistocardiogram, an impedance cardiography, a dispersion based electrocardiography, a respiration sensor, an emotion detector and the like.

In an embodiment of the present disclosure, the one or more bio-markers associated with each of the one or more users include heart rate, heart rate variability, blood flow, blood pressure, movements due to shifts in central blood mass and myocardial electrophysiological responses, respiration information, emotions, skin conductance, photoplethysmography, oxygen saturation, electrocardiography, electroencephalography, muscle activity, accelerometer, temperature and blood glucose, systolic contraction, systemic resistance, cardiac output and the like.

In an embodiment of the present disclosure, the intensity of pain experienced by each of the one or more users is characterized by one or more biological factors (for example, gender and genetics), one or more psychological factors (for example mood and attention), one or more experimental factors and a duration of measurement of the intensity of pain.

In an embodiment of the present disclosure, the computer implemented method further includes examining the intensity of pain experienced by each of the one or more users. The intensity is examined for each increment between a former pain level and a next pain level. The examining is done for constructing a map for showing the intensity of pain experienced at each of the one or more locations of the body for each of the one or more users.

In an embodiment of the present disclosure, the intensity of pain experienced by each of the one or more users is measured by using at least electrocardiography analysis. The electrocardiography analysis is performed using dispersion analysis under high sampling rate.

In another aspect of the present disclosure, a computer program product is provided. The computer program product includes a non-transitory computer readable medium storing a computer readable program. The computer readable program when executed on a computer causes the computer to perform steps including determining intensity of pain experienced by each of one or more users by placing each of one or more bio-sensors at one or more locations on body of each of the one or more users; analyzing the determined intensity of pain from readings of one or more bio-markers obtained by the placing of each of the one or more bio-sensors at the one or more locations on the body of each of the one or more users; recognizing a correlation between the intensity of pain determined from each of the one or more bio-markers associated with each of the one or more users and the one or more locations of each of the one or more bio-sensors with respect to locus of the pain of each of the one or more users; and generating a pain scale for each of the one or more users. The intensity of pain is determined from the one or more bio-markers associated with each of the one or more users. The analyzing is done for determining sensitivity to pain at the one or more locations on the body of each of the one or more users. The correlation reveals a change in the readings of each of the one or more bio-markers for each of the one or more locations of each of the one or more bio-sensors. The recognizing is done for determining an epicenter of pain for each of the one or more users. The pain scale is generated based on the recognizing of the correlation. The pain scale relates distance between the one or more bio-sensors and the locus of the pain with the change in the readings of each of the one or more bio-markers for each of the one or more locations of each of the one or more bio-sensors.

In an embodiment of the present disclosure, the computer readable program when executed on the computer causes the computer to perform a further step of storing the readings of each of the one or more bio-markers and the generated pain scale for each of the one or more users.

In an embodiment of the present disclosure, the one or more bio-sensors include a ballistocardiogram, an impedance cardiography, a dispersion based electrocardiography, a respiration sensor, an emotion detector and the like.

In an embodiment of the present disclosure, the one or more bio-markers associated with each of the one or more users include heart rate, heart rate variability, blood flow, blood pressure, movements due to shifts in central blood mass and myocardial electrophysiological responses, respiration information, emotions, skin conductance, photoplethysmography, oxygen saturation, electrocardiography, electroencephalography, muscle activity, accelerometer, temperature, blood glucose, systolic contraction, systemic resistance, cardiac output and the like.

In an embodiment of the present disclosure, the intensity of pain experienced by each of the one or more users is characterized by one or more biological factors (for example, gender and genetics), one or more psychological factors (for example, mood and attention), one or more experimental factors, a duration of measurement of the intensity of pain and the like.

In an embodiment of the present disclosure, the computer readable program when executed on the computer causes the computer to perform a further step of examining the intensity of pain experienced by each of the one or more users. The intensity is examined for each increment between a former pain level and a next pain level. The examining is done for constructing a map for showing the intensity of pain experienced at each of the one or more locations of the body for each of the one or more users.

In yet another aspect of the present disclosure, a system for detecting intensity of pain experienced by one or more users is provided. The system includes an application server. Further, the application server includes a processor. The processor runs a pain monitoring application. Further, the pain monitoring application includes a determination module, in a processor, the determination module determines the intensity of pain experienced by each of the one or more users by placing each of one or more bio-sensors at one or more locations on body of each of the one or more users; an analysis module, in the processor, the analysis module analyzes the determined intensity of pain from readings of one or more bio-markers obtained by the placing of each of the one or more bio-sensors at the one or more locations on the body of each of the one or more users; a recognizing engine, in the processor, the recognizing engine recognizes a correlation between the intensity of pain determined from each of the one or more bio-markers associated with each of the one or more users and the one or more locations of each of the one or more bio-sensors with respect to locus of the pain of each of the one or more users; and a generating module, in the processor, the generating module generates a pain scale for each of the one or more users. The intensity of pain is determined from the one or more bio-markers associated with each of the one or more users. The analyzing is done for determining sensitivity to pain at the one or more locations on the body of each of the one or more users. The correlation reveals a change in the readings of each of the one or more bio-markers for each of the one or more locations of each of the one or more bio-sensors. The recognizing is done for determining an epicenter of pain for each of the one or more users. The pain scale is generated based on the recognizing of the correlation. The pain scale relates distance between the one or more bio-sensors and the locus of the pain with the change in the readings of each of the one or more bio-markers for each of the one or more locations of each of the one or more bio-sensors.

In an embodiment of the present disclosure, the system further includes a storage module in the processor. The storage module stores the readings of each of the one or more bio-markers and the generated pain scale for each of the one or more users.

In an embodiment of the present disclosure, the analysis module further examines the intensity of pain experienced by each of the one or more users. The intensity is examined for each increment between a former pain level and a next pain level. The examining is done for constructing a map for showing the intensity of pain experienced at each of the one or more locations of the body for each of the one or more users.

In an embodiment of the present disclosure, the one or more bio-sensors include a ballistocardiogram, an impedance cardiography, a dispersion based electrocardiography, a respiration sensor, an emotion detector and the like.

In an embodiment of the present disclosure, the one or more bio-markers associated with each of the one or more users include heart rate, heart rate variability, blood flow, blood pressure, movements due to shifts in central blood mass and myocardial electrophysiological responses, respiration information, emotions, skin conductance, photoplethysmography, oxygen saturation, electrocardiography, electroencephalography, muscle activity, accelerometer, temperature, blood glucose, systolic contraction, systemic resistance, cardiac output and the like.

In an embodiment of the present disclosure, the intensity of pain experienced by each of the one or more users is characterized by one or more biological factors, one or more psychological factors, one or more experimental factors, a duration of measurement of the intensity of pain and the like.

In an embodiment of the present disclosure, the intensity of pain experienced by each of the one or more users is measured by using at least electrocardiography analysis. The electrocardiography analysis is performed using dispersion analysis under high sampling rate.

### BRIEF DESCRIPTION OF THE FIGURES

Having thus described the disclosure in general terms, reference will now be made to the accompanying drawings, which are not necessarily drawn to scale, and wherein:

FIG. 1 illustrates a system showing interaction among various components for detecting pain of users, in accordance with various embodiments of the present disclosure;

FIG. 2 illustrates a block diagram of a communication device, in accordance with various embodiments of the present disclosure; and

FIG. 3 illustrates a flowchart for detecting an intensity of the pain experienced by a user, in accordance with various embodiments of the present disclosure.

### DETAILED DESCRIPTION

It should be noted that the terms “first”, “second”, and the like, herein do not denote any order, quantity, or importance, but rather are used to distinguish one element from another. Further, the terms “a” and “an” herein do not denote a limitation of quantity, but rather denote the presence of at least one of the referenced item.

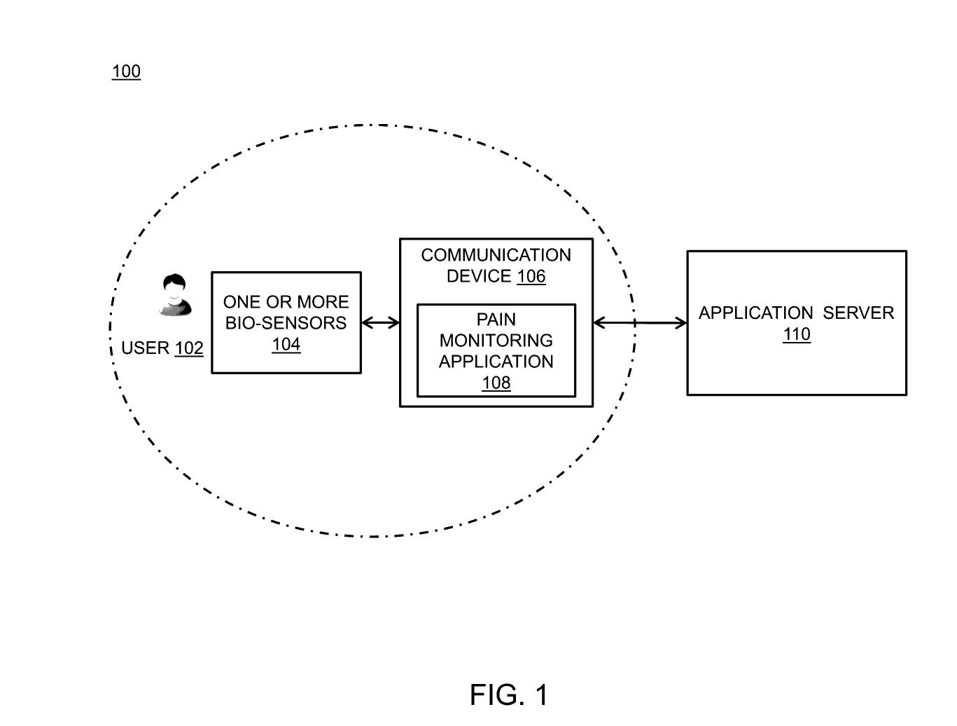

FIG. 1 illustrates a system 100 showing interaction among various components for detecting pain of users, in accordance with various embodiments of the present disclosure. The system 100 includes one or more bio-sensors 104 and a communication device 106 associated with a user 102. Examples of the communication device 106 include but may not be limited to mobile phone, laptop, desktop computer, PDA and the like. The communication device 106 executes a pain monitoring application 108. The pain monitoring application 108 monitors the pain of the user 102 and allows tailoring of treatments accordingly. The pain monitoring application 108 communicates with an application server 110 via a network.

The one or more bio-sensors 104 fetches one or more bio-markers associated with the user 102 including systolic contraction, systemic resistance, cardiac output, heart rate, heart rate variability (HRV), blood flow, blood pressure, movements due to shifts in central blood mass, myocardial electrophysiological responses, respiration information, emotions, skin conductance, photoplethysmography (PPG), oxygen saturation, electrocardiography (ECG), electroencephalography (EEG), muscle activity (EMG), accelerometer, EOG, temperature, blood glucose and the like. In an embodiment of the present disclosure, the one or more bio-sensors 104 may be embedded in a wearable component or a device held close to the user 102. The one or more bio-sensors 104 include but may not be limited to a ballistocardiogram, an impedance cardiography, a dispersion based electrocardiography, a respiration sensor and an emotion detector.

Variability in the one or more bio-markers associated with the user 102 corresponds to autonomic reactions. Examples of the variability in the one or more bio-markers include but may not be limited to change in the systolic contraction, change in the systemic resistance, variation in the cardiac output, the heart rate, the heart rate variability (HRV), change in the blood flow, variation in the blood pressure, the movements due to shifts in central blood mass, change in the myocardial electrophysiological responses, change in the respiration information, change in the photoplethysmography (PPG) readings, change in the electrocardiography (ECG) readings, change in the electroencephalography (EEG) readings, the muscle activity (EMG), the accelerometer, the EOG, the temperature, the blood glucose, 3-axis accelerometer, 3-axis gyroscope and the like. These movements are picked up sensitively by the 3-axis accelerometer but may not be picked up as accurately by naked human eye. The angular part of movement is picked by the 3-axis gyroscope. In an embodiment, the accelerometers show greater movement as the user 102 becomes more restless under pain. In another embodiment, the accelerometers show greater movement when placed near pain area of the user 102.

For example, a user X wears a bio-sensor A of the one or more bio-sensors 104 on upper portion of his left arm (area of pain) and a bio-sensor B on hand of the left arm. The bio-sensor A and the bio-sensor B measures the heart rate, the heart rate variability (HRV), the blood flow and the blood pressure of the user X, however the bio-sensor A shows greater changes in the readings of the bio-markers (the heart rate, the heart rate variability (HRV), the blood flow and the blood pressure) than the readings shown by the bio-sensor B. The bio-sensor A is estimated to show a correct reading as it is placed near to and/or on the pain area.

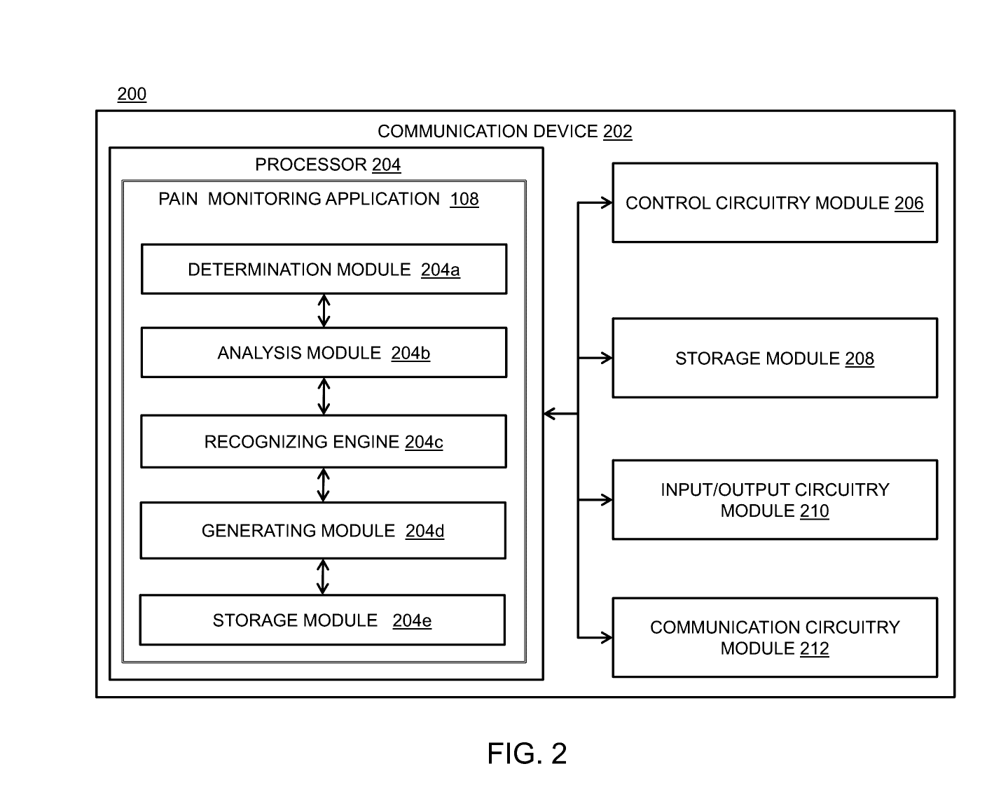

Further, the pain monitoring application 108 generates a pain scale for the user 102 by assessing intensity of the pain of the user 102 from the one or more bio-markers by utilizing the one or more bio-sensors 104 (elaborated in detailed description of FIG. 2). The pain scale shows the intensity of the pain experienced by the user 102 based on distance between the one or more bio-sensors 104 and the area of the pain. Continuing with the above stated example, the pain monitoring application 108 generates the pain scale for the user X which shows different intensities of pain experienced by the user X from the readings of the bio-sensor A and the bio-sensor B.

The application server 110 runs the pain monitoring application 108. A physician may treat the pain of the user 102 by utilizing the pain scale.

It may be noted that in FIG. 1, the pain monitoring application 108 detects the pain of the user 102; however those skilled in the art would appreciate that the pain monitoring application 108 can detect the pain of more than one user. It may also be noted that the user 102 is associated with the communication device 106; however those skilled in the art would appreciate that the user 102 can be associated with more than one communication device having the pain monitoring application 108 installed in it.

FIG. 2 illustrates a block diagram of the communication device 202, in accordance with various embodiments of the present disclosure. It may be noted that to explain the system elements of FIG. 2, references will be made to the system elements of FIG. 1. The communication device 202 includes a processor 204, a control circuitry module 206, a storage module 208, an input/output circuitry module 210 and a communication circuitry module 212. Further, the processor 204 includes a determination module 204a, an analysis module 204b, a recognizing engine 204c, a generating module 204d and a storage module 204e. The above stated components of the processor 204 enable working of the pain monitoring application 108.

The determination module 204a determines the intensity of pain experienced by the user 102 by placing each of the one or more bio-sensors 104 at one or more locations on body of the user 102. The intensity of pain is determined from the one or more bio-markers associated with the user 102. The one or more bio-sensors 104 include but may not be limited to the ballistocardiogram, the impedance cardiography, the dispersion based electrocardiography, the respiration sensor and the emotion detector. The one or more bio-markers that helps in assessing the intensity of the pain of the user 102 include the heart rate (HR), the heart rate variability (HRV), the skin conductance, the respiration information, the blood pressure, the photoplethysmography (PPG), the oxygen saturation, the electrocardiography (ECG), the electroencephalography (EEG), the muscle activity (EMG), restlessness and the like (as illustrated in detailed description of FIG. 1).

The analysis module 204b analyzes the determined intensity of pain from readings of the one or more bio-markers obtained by placing each of the one or more bio-sensors 104 at the one or more locations on the body of each of the user 102. Further, the analysis module 204b examines the intensity of pain experienced by the user 102. Moreover, the analyzing is done for determining sensitivity to pain at the one or more locations on the body of the user 102. The intensity is examined for each increment between a former pain level and a next pain level. Furthermore, the examining is done for constructing a map for showing the intensity of pain experienced at each of the one or more locations of the body for the user 102.

The recognizing engine 204c recognizes a correlation between the intensity of the pain determined from the one or more bio-markers associated with the user 102 and the one or more locations of the one or more bio-sensors 104 with respect to locus of the pain of the user 102. The correlation reveals a relative change in the readings of each of the one or more bio-markers for each of the one or more locations of each of the one or more bio-sensors 104. Moreover, the recognizing is done for determining an epicenter of pain for the user 102. In an embodiment of the present disclosure, the correlation reveals a relative change in readings of each of the one or more bio-markers for each of the one or more locations of a bio-sensor of the one or more bio-sensors 104.

Continuing with the above stated example, the recognizing engine 204c recognizes that the readings of the heart rate, the heart rate variability (HRV), the blood flow and the blood pressure of the user X measured by the bio-sensor A worn by the user X on the upper portion of his left arm (the area of pain) are different from the readings measured by the bio-sensor B worn by the user X on his hand of the left arm. The recognizing engine 204c recognizes the correlation that the bio-sensor A shows greater intensity of the pain experienced by the user X than the bio-sensor B. Further, if the user X wears the bio-sensor A on his right arm, the recognizing engine 204c recognizes that the readings of the heart rate, the heart rate variability (HRV), the blood flow and the blood pressure of the user X measured by the bio-sensor A are different from the readings measured when the user X wore the bio-sensor A on his left arm. The recognizing engine 204c recognizes the correlation that the bio-sensor A shows greater (actual) intensity of the pain when placed near and/or on the area of the pain.

The generating module 204d generates the pain scale for the user 102. The pain scale is generated based on the recognizing of the correlation. The pain scale relates distance between the one or more bio-sensors 104 and the locus of the pain with the change in the readings of each of the one or more bio-markers for each of the one or more locations of each of the one or more bio-sensors 104. Extending the above stated example, the generating module 204d develops a pain scale for the user X. The pain scale for the user X shows the different intensities of the pain experienced by the user X by relating the distances between the bio-sensor A-B and the pain area.

The storage module 204e stores the readings of each of the one or more bio-markers and the generated pain scale for the user 102. In an embodiment of the present disclosure, the intensity of pain experienced by the user 102 is characterized by one or more biological factors (for example, gender, genetics and the like), one or more psychological factors (mood, attention and the like), one or more experimental factors, a duration of measurement of the intensity of pain and the like.

In an embodiment of the present disclosure, the intensity of pain experienced by the one or more users is measured by using at least electrocardiography analysis. The electrocardiography analysis is performed using dispersion analysis under high sampling rate.

It may be noted that in FIG. 2, various modules of the pain monitoring application 108 detects the intensity of pain experienced by the user 102; however those skilled in the art would appreciate that the pain monitoring application 108 may have more number of modules to illustrate the working of the pain monitoring application 108.

Going further, the communication device 202 includes any suitable type of portable electronic device. Examples of the communication device 202 include but may not be limited to a personal e-mail device (e.g., a Blackberry™ made available by Research in Motion of Waterloo, Ontario), a personal data assistant (“PDA”), a cellular telephone, a Smartphone, a handheld gaming device, a digital camera, the laptop computer, and a tablet computer. In another embodiment of the present disclosure, the communication device 202 can be a desktop computer.

From the perspective of this disclosure, the control circuitry module 206 includes any processing circuitry or processor operative to control the operations and performance of the communication device 202. For example, the control circuitry module 206 may be used to run operating system applications, firmware applications, media playback applications, media editing applications, or any other application. In an embodiment, the control circuitry module 206 drives a display and process inputs received from a user interface.

From the perspective of this disclosure, the storage module 208 includes one or more storage mediums including a hard-drive, solid state drive, flash memory, permanent memory such as ROM, any other suitable type of storage component, or any combination thereof. The storage module 208 may store, for example, media data (e.g., music and video files), application data (e.g., for implementing functions on the communication device 202).

From the perspective of this disclosure, the input/output circuitry module 210 may be operative to convert (and encode/decode, if necessary) analog signals and other signals into digital data. In an embodiment, the input/output circuitry module 210 may also convert the digital data into any other type of signal and vice-versa. For example, the input/output circuitry module 210 may receive and convert physical contact inputs (e.g., from a multi-touch screen), physical movements (e.g., from a mouse or sensor), analog audio signals (e.g., from a microphone), or any other input. The digital data may be provided to and received from the control circuitry module 206, the storage module 208 or any other component of the communication device 202.

It may be noted that the input/output circuitry module 210 is illustrated in FIG. 2 as a single component of the communication device 202; however those skilled in the art would appreciate that several instances of the input/output circuitry module 210 may be included in the communication device 202.

The communication device 202 may include any suitable interface or component for allowing the user 102 to provide inputs to the input/output circuitry module 210. The communication device 202 may include any suitable input mechanism. Examples of the input mechanism include but may not be limited to a button, keypad, dial, a click wheel, and a touch screen. In an embodiment, the communication device 202 may include a capacitive sensing mechanism, or a multi-touch capacitive sensing mechanism.

In an embodiment, the communication device 202 may include specialized output circuitry associated with output devices such as, for example, one or more audio outputs. The audio output may include one or more speakers built into the communication device 202, or an audio component that may be remotely coupled to the communication device 202.

The one or more speakers can be mono speakers, stereo speakers, or a combination of both. The audio component can be a headset, headphones or ear buds that may be coupled to the communication device 202 with a wire or wirelessly.

In an embodiment, the input/output circuitry module 210 may include display circuitry for providing a display visible to the user 102. For example, the display circuitry may include a screen (e.g., an LCD screen) that is incorporated in the communication device 202.

The display circuitry may include a movable display or a projecting system for providing a display of content on a surface remote from the communication device 202 (e.g., a video projector). In an embodiment, the display circuitry may include a coder/decoder to convert digital media data into the analog signals. For example, the display circuitry may include video Codecs, audio Codecs, or any other suitable type of Codec.

The display circuitry may include display driver circuitry, circuitry for driving display drivers or both. The display circuitry may be operative to display content. The display content can include media playback information, application screens for applications implemented on the electronic device, information regarding ongoing communications operations, information regarding incoming communications requests, or device operation screens under the direction of the control circuitry module 206. Alternatively, the display circuitry may be operative to provide instructions to a remote display.

In addition, the communication device 202 includes the communication circuitry module 212. The communication circuitry module 212 may include any suitable communication circuitry operative to connect to a communication network and to transmit communications (e.g., voice or data) from the communication device 202 to other devices within the communications network. The communication circuitry module 212 may be operative to interface with the communication network using any suitable communication protocol. Examples of the communication protocol include but may not be limited to Wi-Fi, Bluetooth®, radio frequency systems, infrared, LTE, GSM, GSM plus EDGE, CDMA, and quadband.

In an embodiment, the communication circuitry module 212 may be operative to create a communications network using any suitable communications protocol. For example, the communication circuitry module 212 may create a short-range communication network using a short-range communications protocol to connect to other devices. For example, the communication circuitry module 212 may be operative to create a local communication network using the Bluetooth,® protocol to couple the communication device 202 with a Bluetooth,® headset.

It may be noted that the computing device is shown to have only one communication operation; however, those skilled in the art would appreciate that the communication device 202 may include one more instances of the communication circuitry module 212 for simultaneously performing several communication operations using different communication networks. For example, the communication device 202 may include a first instance of the communication circuitry module 212 for communicating over a cellular network, and a second instance of the communication circuitry module 212 for communicating over Wi-Fi or using Bluetooth®.

In an embodiment, the same instance of the communication circuitry module 212 may be operative to provide for communications over several communication networks. In an embodiment, the communication device 202 may be coupled a host device for data transfers, synching the communication device 202, software or firmware updates, providing performance information to a remote source (e.g., providing riding characteristics to a remote server) or performing any other suitable operation that may require the communication device 202 to be coupled to a host device. Several computing devices may be coupled to a single host device using the host device as a server. Alternatively or additionally, the communication device 202 may be coupled to the several host devices (e.g., for each of the plurality of the host devices to serve as a backup for data stored in the communication device 202).

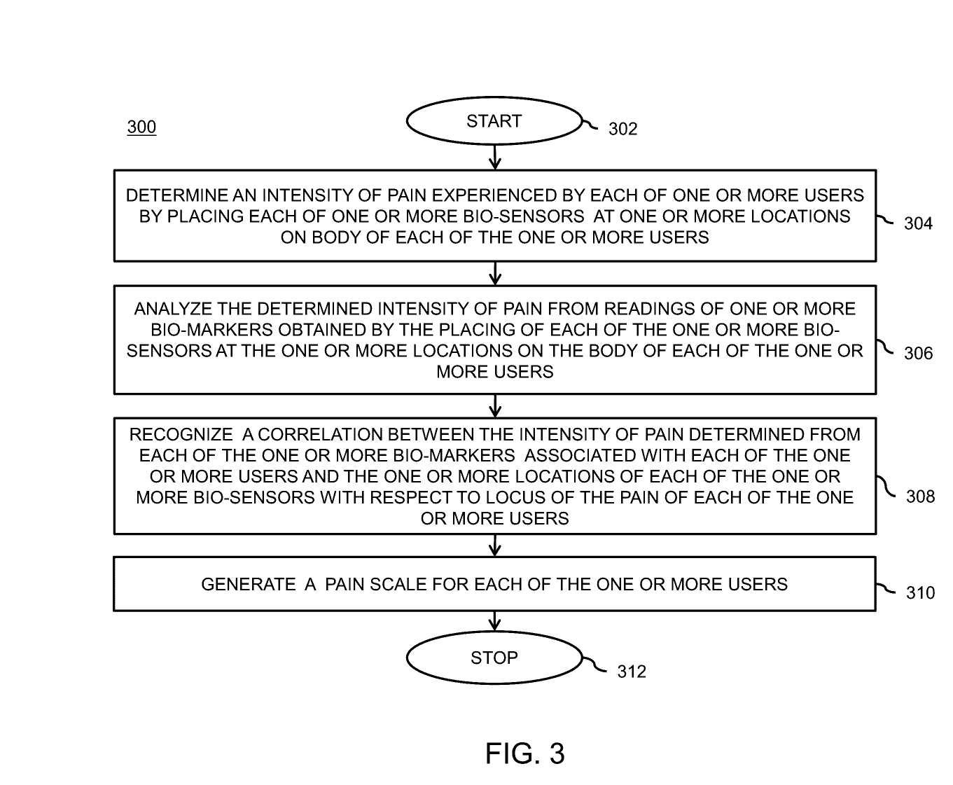

FIG. 3 illustrates a flowchart 300 for detecting the intensity of pain experienced by the user 102, in accordance with various embodiments of the present disclosure. The flow chart 300 initiates at step 302. Following step 302, at step 304, the determination module 204a determines the intensity of pain experienced by the user 102 by placing each of the one or more bio-sensors 104 at the one or more locations on the body of the user 102. The intensity of pain is determined from the one or more bio-markers associated with the user 102. At step 306, the analysis module 204b analyzes the determined intensity of pain from the readings of the one or more bio-markers obtained by the placing of each of the one or more bio-sensors 104 at the one or more locations on the body of the user 102.

At step 308, the recognizing engine 204c recognizes a correlation between the intensity of pain determined from each of the one or more bio-markers associated with the user 102 and the one or more locations of each of the one or more bio-sensors 104 with respect to the locus of the pain of the user 102. The correlation reveals the change in the readings of each of the one or more bio-markers for each of the one or more locations of each of the one or more bio-sensors 104. At step 310, the generating module 204d generates the pain scale for the user 102. The pain scale is generated based on the recognizing of the correlation. The pain scale relates the distance between the one or more bio-sensors 104 and the locus of the pain with the change in the readings of each of the one or more bio-markers for each of the one or more locations of each of the one or more bio-sensors 104. The flowchart 300 terminates at step 312.

It may be noted that the flowchart 300 is explained to have above stated process steps; however those skilled in the art would appreciate that the flowchart 300 may have more/less number of process steps which may enable all the above stated embodiments of the present disclosure.

While the disclosure has been presented with respect to certain specific embodiments, it will be appreciated that many modifications and changes may be made by those skilled in the art without departing from the spirit and scope of the disclosure. It is intended, therefore, by the appended claims to cover all such modifications and changes as fall within the true spirit and scope of the disclosure.

What is claimed is:

1. A computer implemented method for detecting intensity of pain experienced by one or more users, the computer implemented method comprising:
determining, with a processor, the intensity of pain experienced by each of the one or more users by placing each of one or more bio-sensors at one or more locations on body of each of the one or more users, and wherein the intensity of pain being determined from one or more bio-markers associated with each of the one or more users;
analyzing, with the processor, the determined intensity of pain from readings of the one or more bio-markers obtained by the placing of each of the one or more bio-sensors at the one or more locations on the body of each of the one or more users, wherein the analyzing being done for determining sensitivity to pain at the one or more locations on the body of each of the one or more users;
recognizing, with the processor, a correlation between the intensity of pain determined from each of the one or more bio-markers associated with each of the one or more users and the one or more locations of each of the one or more bio-sensors with respect to locus of the pain of each of the one or more users, and wherein the correlation reveals a change in the readings of each of the one or more bio-markers for each of the one or more locations of each of the one or more bio-sensors and wherein the recognizing being done for determining an epicenter of pain for each of the one or more users; and
generating, with the processor, a pain scale for each of the one or more users, wherein the pain scale being generated based on the recognizing of the correlation, and wherein the pain scale relates distance between the one or more bio-sensors and the locus of the pain with the change in the readings of each of the one or more bio-markers for each of the one or more locations of each of the one or more bio-sensors.

2. The computer implemented method as recited in claim 1, further comprising storing the readings of each of the one or more bio-markers and the generated pain scale for each of the one or more users.

3. The computer implemented method as recited in claim 1, wherein the one or more bio-sensors comprises at least one of a ballistocardiogram, an impedance cardiography, a dispersion based electrocardiography, a respiration sensor and an emotion detector.

4. The computer implemented method as recited in claim 1, wherein the one or more bio-markers associated with each of the one or more users comprises heart rate, heart rate variability, blood flow, blood pressure, movements due to shifts in central blood mass and myocardial electrophysiological responses, respiration information, emotions, skin conductance, photoplethysmography, oxygen saturation, electrocardiography, electroencephalography, muscle activity, accelerometer, temperature, blood glucose, systolic contraction, systemic resistance, cardiac output.

5. The computer implemented method as recited in claim 1, wherein the intensity of pain experienced by each of the one or more users being characterized by one or more biological factors, one or more psychological factors one or more experimental factors and a duration of measurement of the intensity of pain.

6. The computer implemented method as recited in claim 1, further comprising examining the intensity of pain experienced by each of the one or more users, wherein the intensity being examined for each increment between a former pain level and a next pain level and wherein the examining being done for constructing a map for showing the intensity of pain experienced at each of the one or more locations of the body for each of the one or more users.

7. The computer implemented method as recited in claim 1, wherein the intensity of pain experienced by each of the one or more users being measured by using at least electrocardiography analysis, wherein the electrocardiography analysis being performed using dispersion analysis under high sampling rate.

8. A computer program product comprising a non-transitory computer readable medium storing a computer readable program, wherein the computer readable program when executed on a computer causes the computer to perform steps comprising:
determining intensity of pain experienced by each of one or more users by placing each of one or more bio-sensors at one or more locations on body of each of the one or more users, and wherein the intensity of pain being determined from one or more bio-markers associated with each of the one or more users;
analyzing the determined intensity of pain from readings of the one or more bio-markers obtained by the placing of each of the one or more bio-sensors at the one or more locations on the body of each of the one or more users, wherein the analyzing being done for determining sensitivity to pain at the one or more locations on the body of each of the one or more users;
recognizing a correlation between the intensity of pain determined from each of the one or more bio-markers associated with each of the one or more users and the one or more locations of each of the one or more bio-sensors with respect to locus of the pain of each of the one or more users, and wherein the correlation reveals a change in the readings of each of the one or more bio-markers for each of the one or more locations of each of the one or more bio-sensors and wherein the recognizing being done for determining an epicenter of pain for each of the one or more users; and
generating a pain scale for each of the one or more users, wherein the pain scale being generated based on the recognizing of the correlation, and wherein the pain scale relates distance between the one or more bio-sensors and the locus of the pain with the change in the readings of each of the one or more bio-markers for each of the one or more locations of each of the one or more bio-sensors.

9. The computer program product as recited in claim 8, wherein the computer readable program when executed on the computer causes the computer to perform a further step of storing the readings of each of the one or more bio-markers and the generated pain scale for each of the one or more users.

10. The computer program product as recited in claim 8, wherein the one or more bio-sensors comprises at least one of a ballistocardiogram, an impedance cardiography, a dispersion based electrocardiography, a respiration sensor and an emotion detector.

11. The computer program product as recited in claim 8, wherein the one or more bio-markers associated with each of the one or more users comprises heart rate, heart rate variability, blood flow, blood pressure, movements due to shifts in central blood mass and myocardial electrophysiological responses, respiration information, emotions, skin conductance, photoplethysmography, oxygen saturation, electrocardiography, electroencephalography, muscle activity, accelerometer, temperature, blood glucose, systolic contraction, systemic resistance, cardiac output.

12. The computer program product as recited in claim 8, wherein the intensity of pain experienced by each of the one or more users being characterized by one or more biological factors, one or more psychological factors, one or more experimental factors and a duration of measurement of the intensity of pain.

13. The computer program product as recited in claim 8, wherein the computer readable program when executed on the computer causes the computer to perform a further step of examining the intensity of pain experienced by each of the one or more users, wherein the intensity being examined for each increment between a former pain level and a next pain level and wherein the examining being done for constructing a map for showing the intensity of pain experienced at each of the one or more locations of the body for each of the one or more users.

14. A system for detecting intensity of pain experienced by one or more users, the system comprising:
an application server, wherein the application server further comprises a processor, the processor being configured to run a pain monitoring application, wherein the pain monitoring application further comprises:
a determination module, in the processor, the determination module being configured to determine the intensity of pain experienced by each of the one or more users by placing each of one or more bio-sensors at one or more locations on body of each of the one or more users, and wherein the intensity of pain being determined from one or more bio-markers associated with each of the one or more users;
an analysis module, in the processor, the analysis module being configured to analyze the determined intensity of pain from readings of the one or more bio-markers obtained by the placing of each of the one or more bio-sensors at the one or more locations on the body of each of the one or more users, wherein the analyzing being done for determining sensitivity to pain at the one or more locations on the body of each of the one or more users;
a recognizing engine, in the processor, the recognizing engine being configured to recognize a correlation between the intensity of pain determined from each of the one or more bio-markers associated with each of the one or more users and the one or more locations of each of the one or more bio-sensors with respect to locus of the pain of each of the one or more users, and wherein the correlation reveals a change in the readings of each of the one or more bio-markers for each of the one or more locations of each of the one or more bio-sensors and wherein the recognizing being done for determining an epicenter of pain for each of the one or more users; and
a generating module, in the processor, the generating module being configured to generate a pain scale for each of the one or more users, wherein the pain scale being generated based on the recognizing of the correlation, and wherein the pain scale relates distance between the one or more bio-sensors and the locus of the pain with the change in the readings of each of the one or more bio-markers for each of the one or more locations of each of the one or more bio-sensors.

15. The system as recited in claim 14, further comprising a storage module in the processor, the storage module being configured to store the readings of each of the one or more bio-markers and the generated pain scale for each of the one or more users.

16. The system as recited in claim 14, wherein the analysis module being further configured to examine the intensity of pain experienced by each of the one or more users, wherein the intensity being examined for each increment between a former pain level and a next pain level and wherein the examining being done for constructing a map for showing the intensity of pain experienced at each of the one or more locations of the body for each of the one or more users.

17. The system as recited in claim 14, wherein the one or more bio-sensors comprises at least one of a ballistocardiogram, an impedance cardiography, a dispersion based electrocardiography, a respiration sensor and an emotion detector.

18. The system as recited in claim 14, wherein the one or more bio-markers associated with each of the one or more users comprises heart rate, heart rate variability, blood flow, blood pressure, movements due to shifts in central blood mass and myocardial electrophysiological responses, respiration information, emotions, skin conductance, photoplethysmography, oxygen saturation, electrocardiography, electroencephalography, muscle activity, accelerometer, temperature, blood glucose, systolic contraction, systemic resistance, cardiac output.

19. The system as recited in claim 14, wherein the intensity of pain experienced by each of the one or more users being characterized by one or more biological factors, one or more psychological factors one or more experimental factors and a duration of measurement of the intensity of pain.

20. The system as recited in claim 14, wherein the intensity of pain experienced by each of the one or more users being measured by using at least electrocardiography analysis, wherein the electrocardiography analysis being performed using dispersion analysis under high sampling rate.
